(19) United States
(12) Patent Application Publication
(10) Pub. No.: US 2008/0092435 A1
(43) Pub. Date: Apr. 24, 2008

(54) METHODS OF PURIFYING BIODIESEL FUELS

(76) Inventors: Justin Bzdek, Fort Collins, CO (US); John Pellegrino, Boulder, CO (US)

Correspondence Address: SHERIDAN ROSS PC, 1560 BROADWAY, SUITE 1200, DENVER, CO, 80202, US

(21) Appl. No.: 11/877,660

(22) Filed: Oct. 23, 2007

Related U.S. Application Data
(60) Provisional application No. 60/862,579, filed on Oct. 23, 2006.

(51) Int. Cl. C10L 1/18; C10L 1/32; G01N 33/22

(52) U.S. Cl. 44/301; 44/307; 73/61.63

(57) ABSTRACT

The invention provides methods of removing chemical species likely to lead to fuel filter plugging from a biodiesel fuel. The invention also provides biodiesel fuels and fuel blends made by these methods. Additionally, the invention provides methods of testing a biodiesel fuel for the presence of these chemical species and evaluating the quality of the fuel and its propensity to plug fuel filters based on the results of this testing.

### CROSS REFERENCE TO RELATED APPLICATION

This application claims the benefit of priority to U.S. Provisional Patent Application No. 60/862,579, filed Oct. 23, 2006, which is incorporated herein by reference.

### FIELD OF THE INVENTION

The invention is directed to improved biodiesel fuel and biodiesel fuel blends and to methods of making and testing these improved fuels.

### BACKGROUND OF THE INVENTION

Throughout much of the twentieth century the U.S. was able to depend on ample domestic supplies of petroleum, however, domestic oil production in the contiguous states peaked in 1970 and has been declining ever since. The U.S. economy relies heavily on diesel-powered vehicles for transportation of people and goods, and diesel fuel constitutes more than 25% of the nation's total fuel use. Diesel engines provide the power to move 94% of all freight in the U.S. as well as 95% of all transit buses and heavy construction machinery. Combining these uses alone, it is currently estimated that the nation consumes more than 90,000 gallons of diesel fuel every minute. There is a need for the U.S. to develop renewable alternatives to this large diesel fuel consumption to diversify the available alternatives to imported petroleum fuels and improve the environmental impact of the national fuel consumption.

Biodiesel is such a renewable and domestically produced diesel fuel alternative that directly displaces, and has a lower environmental impact than petroleum diesel fuel. Biodiesel can be produced from any triglyceride oil and blended with diesel fuel in any proportion. In addition, biodiesel is currently the most effective liquid fuel form that can be derived from the most abundant natural resource, sunlight, as evidenced by its excellent energy balance—biodiesel yields 3.2 units of fuel product energy for every unit of fossil energy consumed in its life cycle.

Unfortunately, a technical difficulty has arisen that continues to plague the biodiesel industry. Marketplace experience shows that biodiesel fuels meeting the total glycerin specification (0.24% mass) and having favorable cold flow properties (cloud point, cold filter plugging point), both neat, and when blended with other fuels, intermittently cause fuel and dispenser filter plugging as well as sedimentation of gelatinous masses in shipping and storage containers. Conventional dead-end filtration has proven ineffective in preventing the formation of these gelatinous masses. Specifically, despite initial filtration through 5-micron filters before blending with diesel fuel, and 10-micron filtration after blending, vehicle fuel filters exposed to the filtered biodiesel fuels have plugged with material. Unexpected fuel filter clogging causes expensive downtime for fleet managers and private vehicle owners, and makes the operation of the vehicles unreliable, which causes unacceptable business or service interruptions.

Therefore, there is an urgent need for biodiesel products that do not suffer from fuel filter plugging problems and reliable methods of forming such biodiesel fuels and biodiesel blends. Biodiesel fuels and blends able to meet the required purity specifications without forming sediments that lead to fuel filter plugging are needed to realize the full potential of the biodiesel industry and biodiesel fuels as renewable and reliable alternatives to diesel fuel.

### SUMMARY OF THE INVENTION

The present invention overcomes the problems of the biodiesel industry described above by providing biodiesel fuels prepared by removing deleterious chemical species from the fuel to insure the filterability of the fuel, both neat and in various biodiesel fuel blends. The purification is accomplished using commercially available, modular membrane separation.

One embodiment of the invention is a method of forming an improved biodiesel fuel by passing a biodiesel stream through a filter to produce an improved biodiesel product. The biodiesel fuel may be a pure biodiesel fuel or a biodiesel fuel blend, such as a blend of biodeisel fuel and a petroleum fuel. The filter of this embodiment may have a molecular weight cut-off (MWCO) of less than about 1,000,000 g/mol, or more preferably, the filter has a molecular weight cut-off between about 50 g/mol and about 1,000,000 g/mol, or even more preferably between about 1000 g/mol and about 250,000 g/mol. The filter may be either an ultrafiltration or a nanofiltration membrane, and is preferably a hydrophilic membrane that may be composed of materials including polysulfones, cellulose acetate, and/or polyvinylidenes. The filter membrane may be formatted for use as a spiral wound module, a hollow fiber membrane, a tubular membrane or a flat sheet membrane. During the filtration, the biodiesel stream is pressurized to maintain a transmembrane operating pressure across the filter between about 0.1 atmospheres to about 100 atmospheres. The biodiesel stream is also preferably maintained at an elevated temperature range between about 15° C. and about 100° C. during the filtration process.

In one preferred embodiment, the biodiesel stream is passed through the filter in a crossflow filtration process. In a related embodiment, the retentate from the filtration process is captured from the filter and returned the biodiesel stream. The filter may be periodically flushed or cleaned with a solvent, such as an alcohol or heptane. Similarly, the filter may be periodically backflushed with the filter permeate to clean or dislodge compounds and complexes that may have accumulated on a surface of the filter.

Another embodiment of the invention is a biodiesel fuel that has a concentration of surface active agents that is less than the concentration of an ASTM-spec B100 biodiesel fuel. Preferably, this biodiesel fuel has a concentration of surface active agents that is less than half the concentration found in an ASTM-spec B100 biodiesel fuel, and more preferably, the concentration of surface active agents in the fuel is less than 10%, or even less than 1% of the concentration of a ASTM-spec B100 biodiesel fuel.

A related embodiment of the invention is an improved biodiesel fuel product formed by the process of removing surface active agents from a biodiesel fuel stream to produce an improved biodiesel fuel. This improved biodiesel fuel product may be formed by passing a biodiesel stream through a filter to produce an improved biodiesel fuel.

Another embodiment of the invention provides a method of testing a biodiesel fuel by cooling the biodiesel fuel to be analyzed to about 4° C. and subjecting the cooled biodiesel fuel to vacuum filtration through a filtration medium while recording the time of filtration and evaluating the quality of the fuel based on the recorded time of filtration.

### BRIEF DESCRIPTION OF THE DRAWINGS

FIG. 1 shows a schematic of a preferred crossflow filtration membrane process of the present invention for the production of refined biodiesel fuels.

FIG. 2 shows the cumulative permeance versus filtration time during crossflow filtration of a several biodiesel samples tested as described in Example 1 of this disclosure.

FIG. 3 shows the cumulative permeance versus amount permeated per unit area (equivalent to filtration time) during crossflow batch filtration of two different biodiesel feedstocks with a Koch M180 ultrafiltration membrane.

FIG. 4 shows the cumulative permeance versus amount permeated per unit area (equivalent to filtration time) during crossflow batch filtration of two biodiesel feedstocks with a GE EWH ultrafiltration membrane, including periodic cleaning steps.

### DETAILED DESCRIPTION OF THE INVENTION

Conventional biodiesel production includes the transesterification of a fat or oil to produce a mixture of primarily long chain methy or ethyl esters of fatty acids and free glycerol, with lesser amounts of mono-, di- and tri-glycerides. Additionally, some oil sources, principally vegetable oils, contain small amounts of sterol glucosides (carbohydrate units glycosidically linked to the hydroxyl group of plant sterols). The free glycerin is largely removed through water washing, while the unreacted or partially-reacted transesterification products (i.e., mono-, di-, and tri-glycerides) as well as lipophilic contaminants, including sterol glucosides, remain in the biodiesel product.

Instances of fuel filter plugging in engines utilizing biodiesel fuels have previously been attributed to the increased detergent properties of biodiesel blends compared to petroleum diesel, since biodiesel blends can dissolve and remove petroleum-diesel residues from fuel tanks and fuel line systems. While this explanation is correct in some instances, it does not explain why vehicles continue to have unexpected fuel filter plugs after vehicles have used a biodeisel blend for an extended period of time.

Microbial growth in the vehicle fuel system is another problem thought to contribute to premature fuel filter plugging. Proper tank maintenance and biocide treatments, however, effectively control microbial growth, but in many instances, microbial contamination has been ruled out as a cause of filter plugging via laboratory tests.

The presence of sterol glucosides in biodiesel fuels has also been proposed as a cause of filter plugging. The sterols are thought to complex together and in combination with any monoglycerides and diglycerides in the fuel to produce aggregates that precipitate out of solution and settle in fuel tanks and clog fuel filters.

The present inventors have collected a variety of plugged fuel filters from multiple vehicle types, including passenger vehicles, light duty trucks, transport trucks, buses and farm equipment, running on a variety of biodiesel blends. GC-MS analysis of these filters showed the plugging material to be primarily monoglycerides. Additionally, sludge material collected from rail cars used to ship ASTM-spec B100 biodiesel was also identified as primarily monoglycerides using GC-MS. These monoglycerides were specifically identified as predominately C16:0 and C18:0 monoglycerides with minor amounts of C14:0, C17:0, C18:0, C20:0, C22:0, and C24:0 monoglycerides, sterol glucosides, phytosterols, glycerin and tocopherols.

The effect of these monoglycerides, which do not normally increase in concentration in biodiesel, can be explained by a process known as reverse micelle formation. In this process, compounds such as monoglycerides behave as surfactants, with a hydrophilic, polar head and a nonpolar tale forming “bubbles” with water at the center and the nonpolar aliphatic tale of each molecule extending out into the hydrophilic biodiesel or biodiesel fuel blend. The components for formation of reverse micelles are present in biodiesel and they are not removed during normal processing or by filtration with filters rated to remove suspended species with a nominal size of 1 μm, because these formative components are smaller than 1 μm. Without intending to be bound by any one theory, it is believed that the micelle components of water and surface active agents (such as bound glycerin present as monoglyceride and diglycerides) aggregate over time, forming larger micelles, which comprise the sediment, haze and sludge found in biodiesel fuels. Increased moisture and decreased temperature will enhance the formation of these reverse micelles and removal of these surfactant components prevents or significantly reduces the formation of mono- and diglyceride-derived micelles that can cause filter plugging by biodiesel fuels. The processes of the present invention remove polar contaminants, including sterol glucosides, mono-acylglycerides, di-acylglycerides, tri-acylglycerides and glycerine that may react alone or in combination with water to create colloidal-sized entities. This includes small crystals and reverse micellar structures, which cause fuel filter plugging and haze formation and gels in bulk biodiesel. This entire class of components is removed or substantially reduced by the methods of the present invention, no matter what level they are present in the untreated biodiesel feed.

The present invention is drawn to methods of removing chemical species present in biodiesel fuels that lead to fuel filter plugging and the biodiesel fuels and biodiesel fuel blends made by these methods. Additionally, the invention provides methods of testing the quality of biodiesel fuels with respect to the potential to plug filters. The present invention provides advanced membrane filtration processes to remove sufficient quantities of mono-, di-, and triglycerides found in biodiesel fuels to reach levels of less than about 0.1% mass of each. These advanced membrane filtration processes result in very low levels of bound glycerin and reduce or eliminate the filter plugging problems experienced with the prior art biodiesel fuels.

Filtration is usually conducted as a dead-end process in which fluid flow passes through the filter in a head-on direction. The fluid flow direction and the permeation through the filter are in parallel. In this arrangement, solutes are predominantly retained in the depths of the filter, though sometimes surface retention is the dominant capture mechanism. All the solutes that are retained by the filter will eventually close off the flow channels, thereby plugging the filter. There are typically no mechanisms for cleaning the plugged filters in these dead-end processes, so the filters must be discarded and replaced.

In contrast, membranes may be more effectively operated continuously in a crossflow process, in which the fluid flows across the surface of the membrane and permeates through the membrane perpendicular to the direction of the incoming fluid flow. Also, in membrane filtration, the solutes that are being rejected by the membrane are retained in the fluid flow and do not permeate the membrane. Thus, crossflow membrane processes are designed to facilitate a clean-in-place strategy with removal of the concentrated solutes. The fluid flow can be recycled in order to recover 100% of the incoming fuel feed.

Therefore, one embodiment of the present invention provides processes that cost-effectively separate surface active agents (primarily monoglycerides and diglycerides) formed in the initial production of biodiesel fuels. These processes include the removal of surfactant species by permeating the biodiesel fuel through a membrane, preferably before reverse micelles have formed. The permeation is preferably conducted through crossflow membrane filtration. Deleterious surface active agents are effectively removed using either or both of nanofiltration and ultrafiltration.

Membranes useful in the processes of the invention are of the category “ultrafiltration,” (UF). These include membranes with a nominal molecular weight cut-off (MWCO; the size of a molecule, at which 90+% of the amount presented to the membrane cannot pass through it) of about 100 g/mol to about 1,000,000 g/mol with a preferred range of about 1000 g/mol to about 250,000 g/mol. The membrane material is preferably a substance considered to be hydrophilic or slightly hydrophilic. Hydrophobic materials are not the preferred type of membrane.

Preferably, the separation is effected immediately following methanolysis (the reaction that creates a biodiesel fuel from a raw oil or fat starting material) although the separation may also be effected before or after storage of the biodiesel fuel or fuel blend, or at the time of dispensing the biodiesel fuel or fuel blend. The separation may even be effected after dispensing of the fuel, that is, within the components of the engine using the fuel but prior to the delivery of the fuel to the fuel filter. The longer the fuel is held between the time of transesterification and the separation of the present invention, the greater will likely be the time and cost of the separation processes, as more reverse micelles will have formed, causing further aggregate formation, leading to the production of more filter-clogging material within the fuel.

These processes may be combined with conventional separation techniques including, but not limited to, settling tanks, adsorptive separations using polar activated carbon, clays, silicas, or other adsorbents in column or packed bed configurations, to selectively adsorb surfactant species. These processes may also include membrane adsorption chromatography to selectively adsorb surfactant species.

In an alternative embodiment of the invention, membrane ultrafiltration may be used to remove reverse micelle aggregates after they have formed in a biodiesel fuel, for example, after prolonged storage or shipping, exposure to cold and/or humid conditions or after fuel filter plugging has arisen in use. In these instances, the remaining fuel may be salvaged by treating the fuel to remove surface active species using the filtration processes described above.

In one embodiment of the invention, a biodiesel fuel is subjected to a membrane separation process to remove glycerides present in the fuel. The membranes utilized in this process may include spiral wound modules, hollow fiber membranes, tubular membranes and/or flat sheet membranes in a plate and frame configuration. Preferably, the process is a feed-and-bleed, crossflow membrane filtration. The successful and economically-viable continuous practice of these processes requires the use of appropriate membranes and filtration conditions, appropriate process configurations, and a viable membrane cleaning protocol.

The molecular mass cutoff for these membrane materials may range between about 50 g/mol and about 1,000,000 g/mol. The biodiesel fuel fluid flow may be subjected to a transmembrane operating pressure between about 0.1 atmospheres to about 100 atmospheres. During filtration, the biodiesel fuel is preferably maintained in a temperature range between about 15° C. and about 100° C. In a preferred embodiment, a membrane with a molecular mass cutoff of about 1000 g/mol, is operated at a transmembrane pressure gradient of about 0.5 atmospheres with a biodiesel fuel at about 30° C.

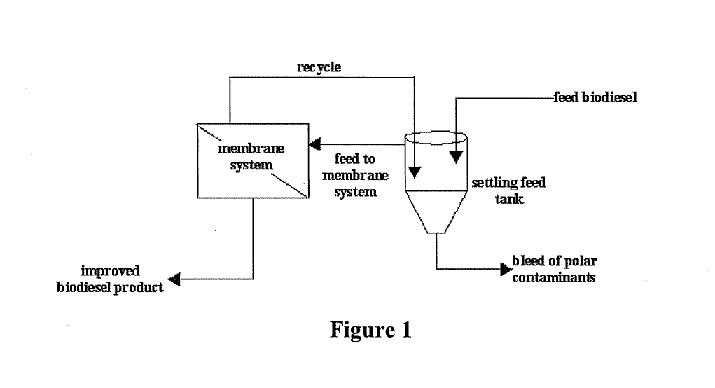

In a preferred embodiment of the invention, the biodiesel fuel flowing past the membrane, which has not permeated the membrane, is recycled to the membrane separation process in a continuous manner to increase utilization of the raw biodiesel fuel. Referring to FIG. 1, this preferred “feed-and-bleed” process may incorporate a number of membrane modules arranged in parallel and series banks. The membrane modules may be spiral wound, plate-and-frame, hollow fiber, or tubular. In a preferred embodiment these modules will be installed with a vertical orientation, that is, the surface of the separating membrane is aligned with the direction of the gravity vector, and the incoming feed to each module will be at the lowest end of the filter. The feed biodiesel enters a settling tank at an intermediate level between the highest point and the beginning of the sloped settling section. The recycle, or non-permeated feed, from the membrane returns to the settling tank at the beginning of the sloped settling section. Thus, the feed to the membrane system will be a combination of “new” feed biodiesel and the recycle stream, and will be withdrawn from the highest liquid level in the tank. Preferably, no agitation is provided within the tank. The permeate passing through the membrane constitutes an improved biodiesel product having a substantially reduced tendency to cause fuel filter plugging or the formation of gelatinous masses upon tank storage. The mass flow rate of this filter permeate (improved biodiesel product) is maintained approximately equal to the mass flow rate of the new feed biodiesel less a small bleed of polar contaminants from the bottom of the sloped settling tank. The ratio of the permeate mass flow rate to the mass flow rate of the feed to the membrane system is maintained in a range between about 5% to about 95%.

Periodic cleaning of the membrane system is required for continuous operation of the separation procedures of the present invention. This cleaning is preferably accomplished by flushing the membrane with either methanol or ethanol in a recirculating fashion without permeation through the membrane. The membrane industry standard practice of backflushing with permeated product (in this case biodiesel) to release accumulated solids may also be performed prior to flushing with either of the preferred alcohol solvents. In preferred embodiments, the alcohol flush stream may be recycled to the biodiesel reactor with or without further purification, as reactor conditions may require.

Another embodiment of the present invention is a biodiesel fuel, either pure or present in a biodiesel fuel blend, produced by a process that separates surface active agents from the biodiesel fuel. Preferably, the fuel of this embodiment has been purified by an ultrafiltration process, a nanofiltration process, or a combination of these filtration processes. Preferably, the fuel has substantially reduced levels of polar species that lead to aggregate formation and fuel filter plugging problems compared to the levels observed in a sample of ASTM-spec biodiesel B100. In one embodiment, the fuel has passed a filtration membrane having a nominal molecular mass cut-off (MWCO) of between 50 g/mol to 1,000,000 g/mol, or more preferably, has passed a membrane with a nominal molecular mass cut-off between 1000 g/mol and 250,000 g/mol. In a related embodiment, the fuel has passed a polysulfone-based filtration membrane with nominal MWCO of 70 kg/mol. In a related embodiment, the fuel has passed a polyvinylidene ultrafiltration membrane. In a related embodiment, the fuel has passed an ultrafiltration membrane with nominal molecular mass cut-off of 100 kg/mol.

Yet another embodiment of the present invention is a biodiesel fuel, either pure or present in a biodiesel fuel blend that significantly reduces or eliminates the formation of fuel filter plugs when the fuel is used in a diesel engine.

Another embodiment of the present invention is a testing method that can be used as a quality-control metric for biodiesel fuels. Using this test process, an aliquot of the biodiesel fuel to be analyzed is refrigerated for at least about 8 hours, and more preferably about 10 hours and more preferably about 12 hours, and even more preferably about 16 hours and most preferably about 24 hours at a temperature between about 0° C. and about 10° C., and more preferably between about 1° C. and about 5° C., and more preferably about 4° C. The sample is then tested for acid, peroxide, aldehyde, mono-, di- and triglyceride, sterol ester and sterol glucoside content or for combinations of these compounds. The tests may be conducted by any of the many known quantitative methodologies for analyzing the content of one or more of these species in the fuel sample. Preferably, the analysis of the sample for one or more of the compounds is conducted by GC-MS. The sample may also be subjected to vacuum filtration through a standard filtration medium and the time of filtration is recorded. Indicia of the likelihood of the tested fuels to cause fuel filter plugging are obtained from the evaluation of the sample using the compound analysis and vacuum filtration tests described above. Fuels identified as having a greater likelihood of causing filter plugging may then be processed to remove surface active compounds from the fuel by the processes described above.

### EXAMPLES

### Biodiesel Feedstocks

Two feedstocks (FS-1 and FS-2) were used for the experimental measurements. Feedstocks 1 and 2 were provided by Blue Sun Biodiesel, Westminster, Colo. Feedstock 1 was a clear “in-specification” material and feedstock 2 contained a “haze.” Both feedstocks were individually mixed before beginning experiments. Both feedstocks had nominal compositions consistent with biodiesel produced from soybean oil. GC-MS analysis of the biodiesel samples used in these process tests consisted of preparing a 25 μL sample dissolved in 1.0 mL of 2-propanol. FAME standards were obtained from Supelco (Bellefonte, Pa., 16823). Standards were prepared in a manner similar to the samples. An Agilent 6890 Gas Chromatograph with 5973 Mass Selective Detector and 7673 Autosampler for two different GC/MS analysis methods. The first method (FAME_{—}100) was used to quantitate the individual FAME components in the samples. All of the samples were then rerun using the second method (FAME_DB5) to get a qualitative measure of monoglycerides and sterols. Table 1 provides details of the equipment and GC/MS conditions used for both the FAME_{—}100 and FAME_DB5 sample analysis. Table 2 shows the results of the GC/MS analysis for the feedstock samples.

TABLE 1
Summary of GC/MS Analysis Methods

| | |
|---|---|
| Method | FAME_100 |
| Column | Varian CP-Select, 100 m × 0.25 mm, 0.25 μm film |
| Carrier Gas | Helium |
| Inlet Conditions | Split mode 100:1, 275° C., Fixed flow |
| | @ 1.00 mL/min |
| Oven | Initial 150° C. for 5.5 min |
| | Ramp 4° C./min to 270° C. (total run time 30.5 min) |
| MS conditions | Scan mode, 33-425 amu. |
| Method | FAME_D5 |
| Column | Agilent HP-5MS, 30 m × 0.25 mm, 0.25 μm film |
| Carrier Gas | Helium |
| Inlet Conditions | Split mode 100:1, 275° C., |
| | Programmed flow Initial 0.90 mL/min for 35 min |
| | Ramp 0.4 mL/min/min to 1.3 mL/min |
| Oven | Initial 120° C. for 1.0 min |
| | Ramp 15° C./min to 165° C.; Hold 0.0 min |
| | Ramp 5° C./min to 240° C.; Hold 0.0 min |
| | Ramp 15° C./min to 300° C.; Hold 22.0 min |
| | (total run time 45 min) |
| MS conditions | Scan mode, 30-500 amu. |

TABLE 2
GC/MS Analysis Results for Feedstock 1 (FS-1) and
Feedstock 2 (FS-2)

Data File Name

| | |
|---|---|
| 0601005.D | 1101011.D |

Sample Name

| | |
|---|---|
| FS-1 | FS-2 |

Vial Number

| | |
|---|---|
| 6 | 11 |

Sample Weight (mg/mL)

| Compound Name | 21.4 | 21.8 |
|---|---|---|
| C8:0 Octanoate | 0.0% | 0.0% |
| C10:0 Caprate | 0.0% | 0.0% |
| C12:0 Laurate | 0.0% | 0.0% |
| C14:0 Myristate | 0.1% | 0.0% |
| C16:0 Palmitate | 11.8% | 11.7% |
| C16:1(9) Palmitoleate | 0.0% | 0.0% |
| C18:0 Sterate | 4.3% | 4.3% |
| 18:1(9) Oleate | 21.5% | 21.7% |
| C18:2(9,12) Linoleate | 54.2% | 53.7% |
| C18:3(9,12,15) Linolenate | 7.6% | 8.0% |
| C20:0 Arachidate | 0.3% | 0.3% |
| C20:1(11) Eicosenoate | 0.0% | 0.0% |
| C22:0 Behenate | 0.3% | 0.3% |
| C22:1(13) Erucate | 0.0% | 0.0% |
| C24:0 Lignocerate | 0.0% | 0.0% |

### Membranes Evaluated

Three separate, commercially available ultrafiltration membranes were used for the production of purified biodiesel test products.
1. EW UF (ultrafiltration membrane based on polysulfone with nominal 70 kg/mol MWCO) commercially available from General Electric. This membrane is a flat sheet format.
2. 3M-PE (polyethylene microfiltration-MF, 68% porosity; 1.7 mil thick; ˜0.19 μm bubble point) commercially available from 3M. This is also a flat sheet format.
3. Koch M180 (polyvinylidene fluoride ultrafiltration membrane with nominal 100 kg/mol MWCO) both flat sheet and tubular formats are commercially available.

### Crossflow Membrane Filtration Tests

Each experiment used a batch filtration approach that started with an initial amount of biodiesel feed (0.3 to 1.5 L) pumped through the membrane test apparatus (across the membrane's surface) with a pressure that was above the external pressure. The biodiesel that permeates through the membrane is the permeate, and is collected at the external pressure. The difference between the feed pressure (supplied by the pump) and the external pressure is called the transmembrane pressure (TMP) and provides the force for “pushing” the fluid through the membrane. The pumping process happened continuously for a period of time and the biodiesel feedstock that did not permeate the membrane was returned back to the feed tank as the experiment progressed. Thus, as the experiment progressed, the actual composition presented to the membrane became more concentrated in retentate. The accumulated volume of permeate divided by the initial feed volume is the recovery, which increases with time. Most experiments were run to recoveries of 30 to 75%.

The filtration figure-of-merit is called the permeance, P/l, which is a design variable. It is calculated as the volume of permeated biodiesel divided by the time of collection, divided by the membrane area, and divided by the nominal pressure drop across the filter (TMP). The higher the permeance, while still producing good product, the more economical is the membrane filtration process because the separation can be operated with lower membrane area and/or lower pumping pressure.

The average permeance decreases with time (or equivalently with cumulative volume permeated) because aggregates that are being removed from the permeated product are becoming more concentrated in the feed to the membrane, and some aggregates are being collected on top of the membrane. This is a typical of crossflow membrane filtration processes.

Several experiments were performed to measure the effects of the filtration conditions on the permeance of the membrane(s). The main effect studied was the TMP. In order to accumulate sufficient permeate to perform the cold soak, modified ASTM 6217 test the permeates from successive experiments were combined in which the TMP was changed in a regular fashion as a process variable.

### Modified ASTM-6217 Cold Soak Test Methods

The filtration time is measured in a standard protocol. The test membrane is a commercial filter with a 47 mm diameter and 1.6 μm nominal pore size (as determined by bubble point tests.) A vacuum filtration is performed with a vacuum of 22-24 in Hg (74 to 81 kPa below atmospheric pressure which is 101.32 kPa at sea level), thus there will be a variation in results depending on the elevation of the test location. A passing filtration time is considered to be 6 minutes (360 s).

### Cold Soak Followed by Flow Filtration Time

1. Prior to running the sample 300 mL of sample is placed in a glass 1 L bottle and set in a bath or refrigerator at about 4.4° C. (40° F.) for 16 hours.
2. After the 16 hour cold soak is completed, the sample is allowed to come back to room temperature on its own without external heating and the sample is tested as soon as possible thereafter.
3. The sample is thoroughly mixed by shaking.
4. The sample is applied to receiving flask, filter and funnel as a unit in a fume hood to minimize operator exposure to fumes.
5. The vacuum pump is started and the vacuum (inches of Hg) after one minute of filtration is recorded. The vacuum must be maintained between 21 and 25 inches of Hg.
6. The sample filtration time is recorded to the nearest 0.5 seconds.
7. If the filtration is not complete after 15 minutes of filtration, the filtration of the complete sample is aborted and the vacuum pressure prior to ceasing the filtration is recorded.
8. The filter membrane and assembly is rinsed with heptane, and the test filter is removed from the filter base using clean forceps.

### Example 1

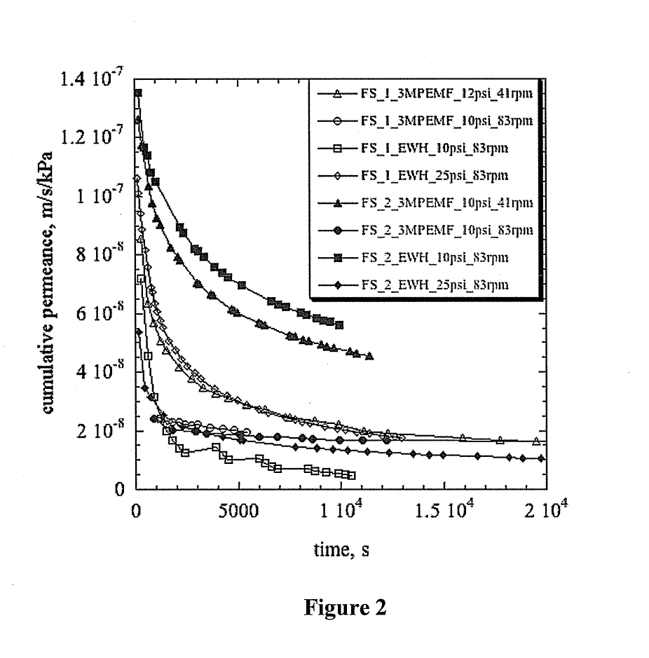

One liter of each well-mixed feed was filtered through flat sheet membranes (3M-PEMF; EW UF) to produce at least 300 mL of permeate and approximately 700 mL of retenate. For each membrane and feedstock combination, 300 mL each of permeate, retentate, and feed were tested for “cold-soak” ASTM 6217 filtration time test. FIG. 2 presents the running average permeance versus time for the crossflow membrane filtration tests.

Table 3 presents the filtration times for the untreated feed, permeate, and retentate (the part of the initial feed not permeated through the membrane—where the removed contaminants are accumulated. Note that neither the nominally “in-spec” biodiesel feed FS_{—}1 nor the hazy FS_{—}2 pass the “cold soak” filtration time test. All permeates through the EW UF membrane passed the filtration time test.

TABLE 3
Modified, ASTM 6217 “cold soak” filtration times for Example 1 crossflow
membrane filtration tests (6:00 min is a “passing” QC test).

| | | modified ASTM- | |
|---|---|---|---|
| | | 6217 “cold-soak” | |
| | | fitration | |
| sample ID | description | (min) | appearance |
| FS-1 | feedstock 1 | 15:00+ | Very pale yellow, clear |
| R_FS_1_3MPEMF | retentate from filtration | not done | Very pale yellow |
| | with 3M MF membrane | | |
| P_FS_1_3MPEMF | permeate from filtration | not done | Very pale yellow |
| | with 3M MF membrane | | |
| R_FS_1_EWHUF | retentate from filtration | 14:30 | Very pale yellow |
| | with EW UF membrane | | |
| P_FS_1_EWHUF | permeate from filtration | 0:24 | Very pale yellow |
| | with EW UF membrane | | |
| FS-2 | feedstock 2 | 15:00+ | Yellow, clear |
| R_FS_2_3MPEMF | retentate from filtration | 15:00+ | Yellow, clear supernate, white |
| | with 3M MF membrane | | sediment; est. 10-15% of sample |
| | | | volume |
| P_FS_2_3MPEMF | permeate from filtration | 14:26 | Yellow |
| | with 3M MF membrane | | |
| R_FS_2_EWHUF | retentate from filtration | 15:00+ | Yellow, clear supernate, trace of |
| | with EW UF membrane | | white sediment |
| P_FS_2_EWHUF | permeate from filtration | 0:30 | Yellow |
| | with EW UF membrane | | |

### Example 2

More than one liter of each well-mixed feedstock was filtered through a flat sheet Koch M180 membrane with TMP pressures increasing sequentially from 34 to 207 kPa (5 to 30 psi). A volume of approximately 165 mL of permeate was collected at each pressure. Samples of the permeates were combined in order to perform triplicate “cold soak” filtration time tests.

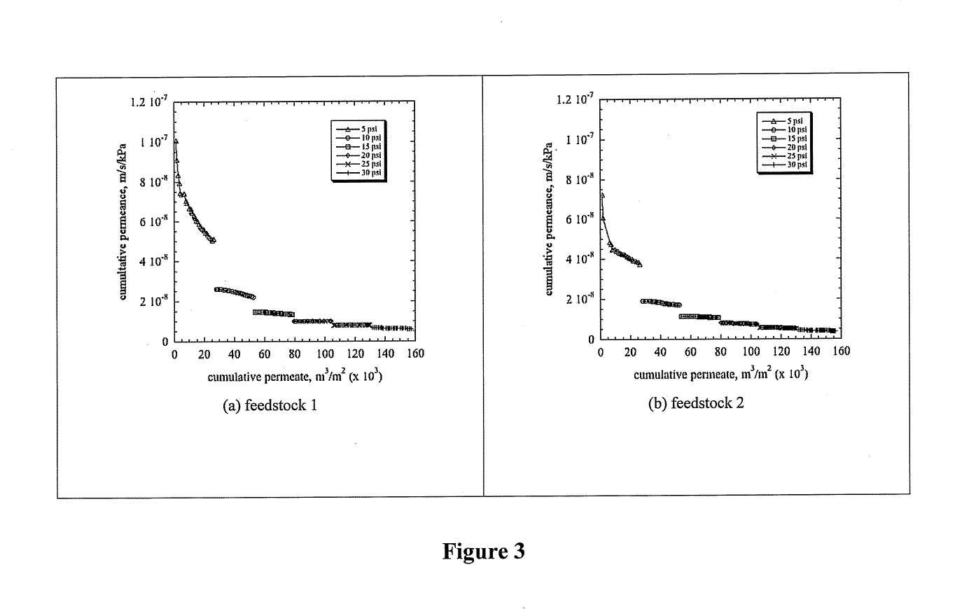

FIG. 3 presents the cumulative permeance versus the amount permeated (divided by the membrane area). This provides the same relationship as a plot versus time but is a more systematic basis of comparison because it directly relates to the amount of contaminants that may be deposited on the membrane. Table 4 shows the results of the cold soak filtration times and the gain in mass of the filtration test filter while performing the ASTM procedures. Neither the “in spec” nor the hazy feedstock passed the test, but the mixed permeate from the filtration trials did.

TABLE 4
Modified, ASTM 6217 “cold soak” filtration times for Example 2 crossflow
membrane filtration tests (6:00 min is a “passing” QC test for 300 mL).

| | | | modified ASTM- | |
|---|---|---|---|---|
| | | volume | 6217 “cold-soak” | deposit on ASTM |
| | | filtered, | fitration | test filter, mg/L |
| sample ID | description | mL | (min) | filtered |
| FS-1_CS | feedstock 1 | 134 | 15:00+ | 167.6 |
| FS-1_CS | feedstock 1 (replicate) | 98 | 15:00+ | 165.8 |
| FS-1_CS | feedstock 1 (replicate) | 94 | 15:00+ | 134.3 |
| P_FS_1_M180_CS | permeate from filtration of | 300 | 0:43 | 39.3 |
| | FS-1 with Koch M180 | | | |
| | membrane | | | |
| P_FS_1_M180_CS | permeate from filtration of | 300 | 0:38 | 20.8 |
| | FS-1 with Koch M180 | | | |
| | membrane (replicate) | | | |
| P_FS_1_M180_CS | permeate from filtration of | 260* | 0:34 | 227.9 |
| | FS-1 with Koch M180 | | | |
| | membrane (replicate) | | | |
| FS-2_CS | feedstock 2 | 14 | 15:00+ | 22835.7 |
| FS-2_CS | feedstock 2 (replicate) | 15 | 15:00+ | 6554.7 |
| FS-2_CS | feedstock 2 (replicate) | 13 | 15:00+ | 6823.8 |
| P_FS_2_M180_CS | permeate from filtration of | 300 | 0:28 | 263.0 |
| | FS-2 with Koch M180 | | | |
| | membrane | | | |
| P_FS_2_M180_CS | permeate from filtration of | 300 | 0:28 | 467.1 |
| | FS-2 with Koch M180 | | | |
| | membrane (replicate) | | | |
| P_FS_2_M180_CS | permeate from filtration of | 300 | 0:28 | 243.4 |
| | FS-2 with Koch M180 | | | |
| | membrane (replicate) | | | |

*sufficient volume for official testing was unavailable due to spillage

### Example 3

More than 1.5 L of well-mixed feedstock 2 was filtered through a EW UF membrane with reservoir pressures increasing sequentially from 5 to 30 psi. A volume of approximately 165 mL of permeate was collected at each pressure. Following this, further filtration tests were performed after various membrane cleaning protocols. That is, six batches were performed with increasing TMP from 5 to 30 psi. Then the filtration was stopped and a small volume (185 mL) of methanol (MeOH) was pumped across the top of the membrane for 30 min. Then the filtration was resumed (1_MeOH test) with same initial charge of fresh feed that had already been concentrated in contaminants, and three pressures steps (5, 15, and 25 psi TMP) were applied to collect 165 mL each of permeate. The filtration was stopped and the same 185 mL of methanol cleaning solution was flushed across the top of the membrane for 30 min. Then the filtration was resumed (2_MeOH test) with same initial charge of fresh feed that had already been concentrated in contaminants, and three pressures steps (5, 15, and 25 psi TMP) were applied to collect 165 mL each of permeate. Then the filtration was stopped and a small volume (185 mL) of ethanol (EtOH) was pumped across the top of the membrane for 30 min. Then the filtration was resumed (2_MeOH_{—}1_EtOH test) with same initial charge of fresh feed that had already been concentrated in contaminants, and two pressures steps (5 and 15 psi TMP) were applied to collect 165 mL each of permeate.

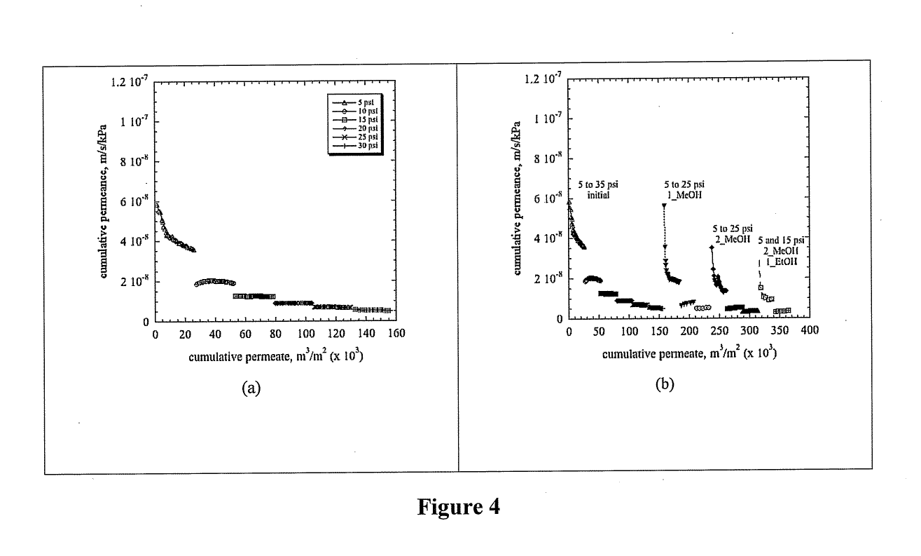

FIG. 4a shows the permeance using the new membrane and feedstock 2. FIG. 4b shows the same data, but includes the subsequent filtration done after flushing with the indicated alcohols. In all cases, the alcohols were effective at regenerating the permeance and cleaning off some or all of the contaminants deposited on the membrane's surface. The continual trend downward in permeance occurs because the initial feedstock sample becomes more concentrated as “clean” permeate biodiesel is removed. Table 5 lists the “cold soak” filtration times for samples from this series of tests. In all cases, the permeate provides an improved biodiesel relative to the starting feedstock 2 and, of course, the non-permeated fluid that is more highly concentrated in the polar contaminants.

TABLE 5
Modified, ASTM 6217 “cold soak” filtration times for Example 3 crossflow
membrane filtration tests (6:00 min is a “passing” QC test for 300 mL).

| | | | modified | |
|---|---|---|---|---|
| | | | ASTM-6217 | deposit on |
| | | volume | “cold-soak” | ASTM test |
| | | filtered, | fitration | filter, mg/L |
| sample ID | description | mL | (min) | filtered |
| FS-2_CS | feedstock 2 | 14 | 15:00+ | 22835.7 |
| FS-2_CS | feedstock 2 (replicate) | 15 | 15:00+ | 6554.7 |
| FS-2_CS | feedstock 2 (replicate) | 13 | 15:00+ | 6823.8 |
| R_FS_2_EWH_0_wash | retentate from filtration | 18 | 15:00+ | 5199.4 |
| | of FS-2 with GE EWH | | | |
| | membrane | | | |
| P_FS_2_EWH_0_wash | permeate from filtration | 300 | 0:28 | 32.3 |
| | of FS-2 with GE EWH | | | |
| | membrane | | | |
| R_FS_2_EWH_1_MeOH | retentate from filtration | 88 | 15:00+ | 1200.9 |
| | of FS-2 with GE EWH | | | |
| | membrane after 1^{st} | | | |
| | methanol flush cleaning | | | |
| P_FS_2_EWH_1_MeOH | permeate from filtration | 300 | 0:26 | 171.2 |
| | of FS-2 with GE EWH | | | |
| | membrane after 1^{st} | | | |
| | methanol flush cleaning | | | |
| R_FS_2_EWH_2_MeOH | retentate from filtration | 17 | 15:00+ | 12656.5 |
| | of FS-2 with GE EWH | | | |
| | membrane after 2^{nd} | | | |
| | methanol flush cleaning | | | |
| P_FS_2_EWH_2_MeOH | permeate from filtration | 300 | 0:28 | 1492.0 |
| | of FS-2 with GE EWH | | | |
| | membrane after 2^{nd} | | | |
| | methanol flush cleaning | | | |
| P_FS_2_EWH_2_MeOH_1_EtOH | permeate from filtration | 300 | 1:09 | 781.7 |
| | of FS-2 with GE EWH | | | |
| | membrane after 2^{nd} | | | |
| | methanol flush cleaning | | | |
| | and 1^{st} ethanol cleaning | | | |

The foregoing description of the present invention has been presented for purposes of illustration and description. The description is not intended to limit the invention to the form disclosed herein. Variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method of forming an improved biodiesel fuel comprising passing a biodiesel stream through a filter having a molecular weight cut-off of less than about 1,000,000 g/mol to produce an improved biodiesel product.

2. The method of claim 1, wherein the filter has a molecular weight cut-off between about 50 g/mol and about 1,000,000 g/mol.

3. The method of claim 1, wherein the filter has a molecular weight cut-off between about 1000 g/mol and about 250,000 g/mol.

4. The method of claim 1, wherein the filter has a molecular weight cut-off of about 100 g/mol.

5. The method of claim 1, wherein the filter has a molecular weight cut-off of about 70 g/mol.

6. The method of claim 1, wherein the filter is an ultrafiltration membrane.

7. The method of claim 1, wherein the filter is a nanofiltration membrane.

8. The method of claim 1, wherein the filter is a hydrophilic membrane.

9. The method of claim 1, wherein the filter is an ultrafiltration membrane comprising a material selected from the group consisting of a polysulfone, cellulose acetate, a polyethylene, and a polyvinylidene.

10. The method of claim 1, wherein the filter is a filter type selected from the group consisting of spiral wound modules, hollow fiber membranes, tubular membranes and flat sheet membranes.

11. The method of claim 1, wherein the biodiesel stream is passed through the filter in a crossflow filtration process.

12. The method of claim 1, wherein the biodiesel stream is pressurized to maintain a transmembrane operating pressure across the filter between about 0.1 atmospheres to about 100 atmospheres.

13. The method of claim 1, wherein the biodiesel stream is maintained in a temperature range between about 15° C. and about 100° C.

14. The method of claim 1, wherein the biodiesel is a blend of biodiesel fuel and a petroleum fuel.

15. The method of claim 1, wherein a retentate on the filter is returned the biodiesel stream.

16. The method of claim 1, further comprising flushing the filter with a solvent.

17. The method of claim 1, further comprising backflushing the filter with a filter permeate.

18. A method of forming an improved biodiesel fuel comprising passing a biodiesel stream through a filter having a membrane with a molecular mass cutoff of about 1000 g/mol, operated at a transmembrane pressure gradient of about 0.5 atmospheres, wherein the biodiesel fuel is maintained at about 30° C.

19. A biodiesel fuel comprising a fuel having a concentration of surface active agents that is less than the concentration of a ASTM-spec B100 biodiesel fuel.

20. The biodiesel fuel of claim 19, wherein the concentration of surface active agents in the fuel is less than half the concentration of a ASTM-spec B100 biodiesel fuel.

21. The biodiesel fuel of claim 19, wherein the concentration of surface active agents in the fuel is less than 10% of the concentration of a ASTM-spec B100 biodiesel fuel.

22. The biodiesel fuel of claim 19, wherein the concentration of surface active agents in the fuel is less than 1% of the concentration of a ASTM-spec B100 biodiesel fuel.

23. An improved biodiesel fuel product formed by a process comprising passing a biodiesel stream through a filter to produce an improved biodiesel product.

24. An improved biodiesel fuel product formed by a process comprising removing surface active agents from a biodiesel fuel stream to produce an improved biodiesel product.

25. A method of testing a biodiesel fuel comprising:
a) cooling a biodiesel fuel to be analyzed to about 4° C.;
b) subjecting the cooled biodiesel fuel to vacuum filtration through a filtration medium while recording the time of filtration; and,
c) evaluating the quality of the fuel based on the recorded time of filtration.
